(19) United States
(12) Patent Application Publication
(10) Pub. No.: US 2021/0379410 A1
(43) Pub. Date: Dec. 9, 2021

(54) PERSONAL AIR STERILIZATION AND PURIFICATION DEVICE

(71) Applicant: Michael Thomas Anthony, Naples, FL (US)

(72) Inventor: Michael Thomas Anthony, Naples, FL (US)

(21) Appl. No.: 16/900,302

(22) Filed: Jun. 12, 2020

(30) Foreign Application Priority Data
Jun. 4, 2020 (CN) 2020105012119

(51) Int. Cl. A62B 7/10; B01D 46/10; B01D 46/00; B01D 46/42; A61L 9/20; A62B 23/02

(52) U.S. Cl. CPC A62B 7/10; B01D 46/10; B01D 46/0005; B01D 46/4245; B01D 46/0028; A61L 2209/12; A62B 23/02; B01D 2265/028; A61L 2209/14; A61L 2209/15; A61L 9/20

(57) ABSTRACT

Provided is a personal air sterilization and purification device, comprising: a purification device body, a filter element, a filter element cover, and a sterilization assembly, a notch being formed at an upper portion of the purification device body, a bottom of the notch being provided with a filter installation slot, a bottom of the filter installation slot communicating with an interior of the purification device body, the filter element cover being snapped onto the purification device body, the filter element cover matching the notch, a filtering space being formed between the filter element cover and the filter installation slot, the filter element being detachably installed at a bottom side of the filtering space, the filter element cover being arranged opposite to the filter element, and the sterilization assembly being fixedly installed inside the filter element cover.

### BACKGROUND OF THE INVENTION

### 1. Field of the Invention

The invention relates to the technical field of air purification, and more particularly, to a personal air sterilization and purification device.

### 2. Description of the Related Art

Air pollution (e.g., smog) rises at an alarming rate, an increasing number of people begin to purchase air purifiers, such as PM 2.5 masks. However, personal air purification devices available now are masks or protective masks. Such devices have poor ventilation performances, which may cause breathing difficulties for some users. Worse still, people often use the masks repeatedly. In doing so, a lot of bacteria and virus may remain on the masks, and a filter layer in the mask fails to absorb the bacteria and virus in a very short period of time, and frequent replacement of masks is a waste of resources, to some extent.

### SUMMARY OF THE INVENTION

In order to solve the foregoing problems exist in the prior art, an object of the present invention is to provide a personal air sterilization and purification device, comprising: a purification device body, a filter element, a filter element cover, and a sterilization assembly, a notch being formed at an upper portion of the purification device body, a bottom of the notch being provided with a filter installation slot, a bottom of the filter installation slot communicating with an interior of the purification device body, the filter element cover being snapped onto the purification device body, the filter element cover matching the notch, a filtering space being formed between the filter element cover and the filter installation slot, the filter element being detachably installed at a bottom side of the filtering space, the filter element cover being arranged opposite to the filter element, and the sterilization assembly being fixedly installed inside the filter element cover.

In a further preferred embodiment, the filter element cover comprises: a ventilation cover and a snap-fit cover, the ventilation cover is integrated with the snap-fit cover, the ventilation cover is provided with a plurality of ventilation holes, an outer edge of the ventilation cover extends vertically and downwardly to form a first edge covering structure, the sterilization assembly is installed at an inner side of the first edge covering structure, an opening is formed at an upper portion of the snap-fit cover, the opening matches a profile of a lower end of the first edge covering structure, the first edge covering structure is disposed on the opening, an outer edge of the snap-fit cover extends vertically and downwardly to form a second edge covering structure, the second edge covering structure has an L-shaped horizontal section, an inner side of the second edge covering structure is provided with a plurality of clamping feet, and inner side of the notch is provided with a plurality of bayonets, and the filter element cover are fitted to the purification device body through the plurality of clamping feet and the plurality of bayonets.

In a further preferred embodiment, wherein the sterilization assembly comprises: a flexible circuit board, a plurality of sterilization units, and a USB cable, the flexible circuit board is arranged along an inner edge of the first edge covering structure, the plurality of sterilization units are uniformly installed on the flexible circuit board, one end of the USB cable passes through the first edge covering structure to connect with the flexible circuit board, the other end of the USB cable is disposed at an outer side of the filter element cover, and the other end of the USB cable is provided with a USB connector.

In a further preferred embodiment, the purification device body comprises: a device main housing, a power supply, a fan assembly, a control circuit board, and an air pipe, the fan assembly is fixedly installed on one side of an interior of the device main housing, the control circuit board is fixedly installed on the other side of the device main housing, the power supply is fixedly installed between the fan assembly and the control circuit board, both the power supply and the fan assembly are connected to the control circuit board, the air pipe is arranged on one side other than the side on which the fan assembly opposites to the power supply, one end of the air pipe passes through the device main housing and communicates with an interior of the fan assembly, and the other end of the air pipe communicates with a breathing apparatus of a user.

In a further preferred embodiment, the device main housing comprises: an upper housing and a lower housing, the upper housing is detachably installed at an upper end of the lower housing, an outer edge of the upper housing matches an outer edge of the lower housing, the notch is formed on a side of an upper portion of the upper housing, a supporting edge is provided at an inner side of the notch in a horizontal direction, an outer edge of the supporting edge matches the outer edge of the filter element, the filter element is placed on the supporting edge, an upper air duct opening is formed on an inner side of the supporting edge, the upper air duct opening directly faces an upper portion of the fan assembly, a mounting opening is disposed at one side of the lower housing, and the mounting opening matches one end of the air pipe.

In a further preferred embodiment, the fan assembly comprises: an upper sealing wall, a side sealing wall, and a turbo fan, the turbo fan is fixedly installed on a side of an upper surface of the lower housing, the side sealing wall is installed at an upper surface of the lower housing in a vertical direction, the turbo fan is enclosed in the side sealing wall, the side sealing wall communicates with the mounting opening, the upper sealing wall is arranged in a horizontal direction, the upper sealing wall is fixedly installed in the side sealing wall, a contour of the upper sealing wall matches a contour of the side sealing wall, a lower air duct opening is formed in the upper sealing wall, an inner edge of the lower air duct opening matches an outer edge of the turbo fan, the lower air duct opening directly faces the upper air duct opening, and an air duct from the filter element to the air pipe is formed among the upper sealing wall, the side sealing wall and the lower housing.

In a further preferred embodiment, the control circuit board is further provided with a USB interface, the USB interface is arranged on one side other than the side on which the control circuit board opposites to the power supply, the USB interface runs through the device main housing, and the USB interface matches the USB connector.

In a further preferred embodiment, the number of the sterilization units is not less than six.

In a further preferred embodiment, the sterilization units are ultraviolet LEDs.

In a further preferred embodiment, a clamping hook is arranged on an outer wall of the main housing close to the USB interface, one end of the USB cable, close to the USB connector, is operatively snapped onto the clamping hook.

By adopting the above-mentioned technical solutions, the present invention has the beneficial effects over the prior art, that is,

1. with the application of the invention, an air purification device is provided, wherein the device has a simple structure and is easy to carry, and is capable of sterilizing the filter element according to user's needs;

2. with the application of the invention, cleanliness of the filter element is improved during its use, the service efficiency of the filter element is further improved, the cleanliness of the interior of the filter element cover is further improved, and a lot of bacteria and virus will not remain in the filter element, thereby bacteria and virus will not contaminate hands or spread to a mouth and ears when replacing the filter element manually.

### BRIEF DESCRIPTION OF THE DRAWINGS

The accompanying drawings, together with the specification, illustrate exemplary embodiments of the present disclosure, and, together with the description, serve to explain the principles of the present invention.

FIG. 1 is schematic view of a personal air sterilization and purification device according to the present invention;

FIG. 2 is an explosive view of a personal air sterilization and purification device according to the present invention;

FIG. 3 is a schematic view of a filter element cover of a personal air sterilization and purification device according to the present invention when viewed from a first perspective;

FIG. 4 is a schematic view of a filter element cover of a personal air sterilization and purification device according to the present invention when viewed from a second perspective; and

FIG. 5 is a schematic view of a sterilization assembly of a personal air sterilization and purification device according to the present invention.

In the accompanying drawings:

1 purification device body; 2 filter element; 3 filter element cover; 4 sterilization assembly; 11 notch; 31 ventilation cover; 32 snap-fit cover; 311 first edge coving structure; 312 ventilation hole; 313 second edge covering structure; 321 clamping foot; 41 flexible circuit board; 42 sterilization unit; 43 USB cable; 44 USB connector; 101 device main housing; 102 power supply; 103 fan assembly; 104 control circuit board; 105 air pipe; 1011 upper housing; 1012 lower housing; 1013 supporting edge; 1014 upper air duct opening; 1031 upper sealing wall; 1032 side sealing wall; 1033 turbo fan; 1035 USB interface; 45 clamping hook.

### DETAILED DESCRIPTION

The present invention will now be described more fully hereinafter with reference to the accompanying drawings and particular embodiments. The invention, however, is not limited to the particular embodiments.

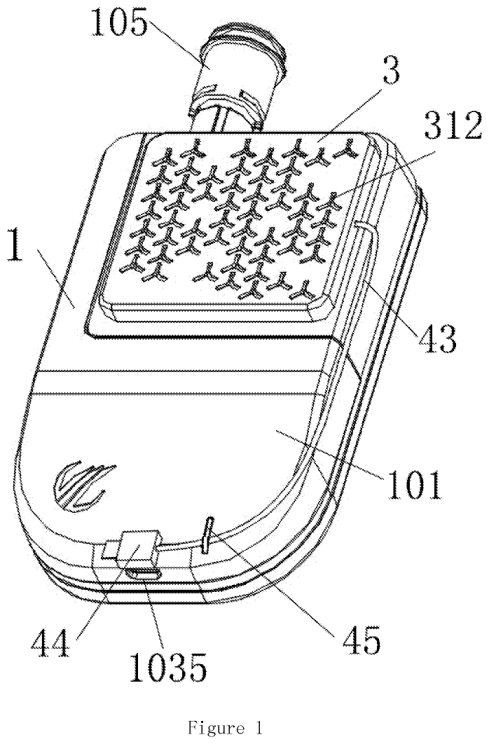

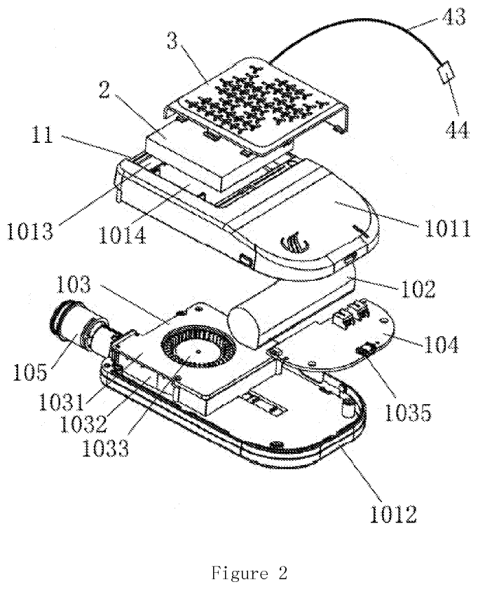

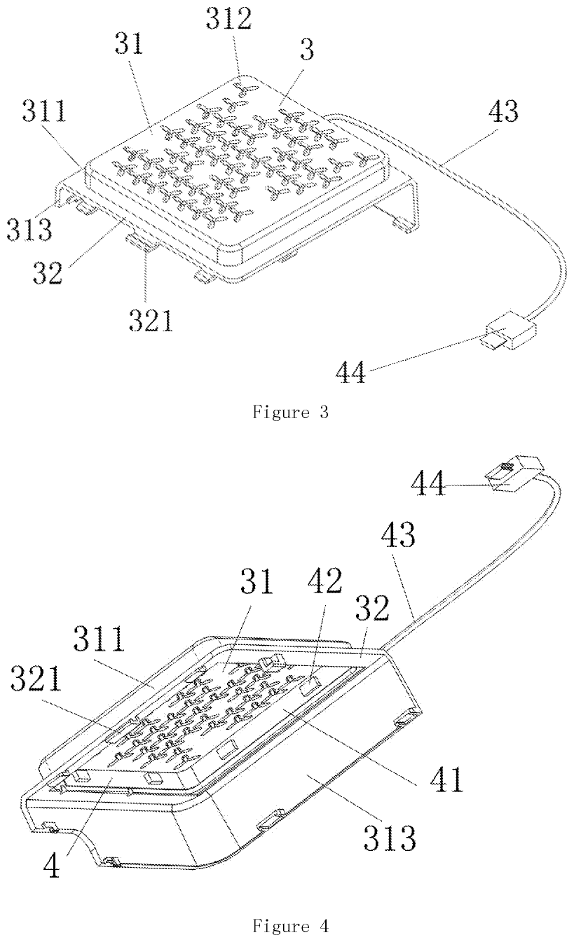

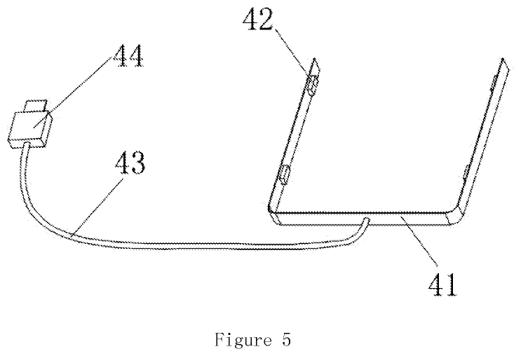

As shown in FIGS. 1 to 5, a personal air sterilization and purification device is provided, comprising: a purification device body 1, a filter element 2, a filter element cover 3, and a sterilization assembly 4, a notch 11 being formed at an upper portion of the purification device body 1, a bottom of the notch 11 being provided with a filter installation slot, a bottom of the filter installation slot communicating with an interior of the purification device body 1, the filter element cover 3 being snapped onto the purification device body 1, the filter element cover 3 matching the notch 11, a filtering space being formed between the filter element cover 3 and the filter installation slot, the filter element 2 being detachably installed at a bottom side of the filtering space, the filter element cover 3 being arranged opposite to the filter element 2, and the sterilization assembly 4 being fixedly installed inside the filter element cover 3. Furthermore, the filter element cover 3 is opened to replace the filter element 2. The sterilization assembly 4 arranged inside the filter element cover 3 operates according to the users' needs, such that a surface of the filter element 2 and the interior of the filter element cover 3, in which a lot of bacteria and virus may be accumulated when filtering air, are sterilized.

Furthermore, as a preferred embodiment, the filter element cover 3 comprises: a ventilation cover 31 and a snap-fit cover 32, the ventilation cover 31 is integrally formed with the snap-fit cover 32, the ventilation cover 31 is provided with a plurality of ventilation holes 312, an outer edge of the ventilation cover 31 extends vertically and downwardly to form a first edge covering structure 311, the sterilization assembly 4 is installed at an inner side of the first edge covering structure 311, an opening is formed at an upper portion of the snap-fit cover 32, the opening matches a profile of a lower end of the first edge covering structure 311, the first edge covering structure 311 is disposed on the opening, an outer edge of the snap-fit cover 32 extends vertically and downwardly to form a second edge covering structure 313, the second edge covering structure 313 has an L-shaped horizontal section, an inner side of the second edge covering structure 313 is provided with a plurality of clamping feet 321, and inner side of the notch 11 is provided with a plurality of bayonets, and the filter element cover 3 are fitted to the purification device body 1 through the plurality of clamping feet 321 and the plurality of bayonets. Furthermore, the plurality of ventilation holes 312 may preferably be Y-shaped; the presence of the first edge covering structure 311 and the second edge covering structure 313 allows the sterilization assembly 4 and the filter element 2 to keep a certain distance, so that it is easier for the sterilization assembly 4 to perform sterilization operation; with force applied to the filter element cover 3, the filter element cover 3 may be slightly slid to the side of the notch 11 to release the clamping feet 321 from the bayonets, so as to open the filter element cover 3.

Furthermore, as a preferred embodiment, the sterilization assembly 4 comprises: a flexible circuit board 41, a plurality of sterilization units 42, and a USB cable 43, the flexible circuit board 41 is arranged along an inner edge of the first edge covering structure 311, the plurality of sterilization units 42 are uniformly installed on the flexible circuit board 41, one end of the USB cable 43 passes through the first edge covering structure 311 to connect with the flexible circuit board 41, the other end of the USB cable 43 is disposed at an outer side of the filter element cover 3, and the other end of the USB cable 43 is provided with a USB connector 44. Furthermore, the USB cable 43 is connected to the power supply 102 or an external power supply to power the flexible circuit board 41, then the plurality of sterilization units 42 start to sterilize air. Furthermore, the flexible circuit board 41 preferably comprises, which are arranged in series, a first circuit board segment, a second circuit board segment, and a third circuit board segment, in which the first circuit board segment, the second circuit board segment, and the third circuit board segment are integrated. Both the first circuit board segment and the third circuit board segment are perpendicular to the second circuit board segment. The joints between the first circuit board segment and the second circuit board segment, or between the third circuit board segment and the second circuit board segment are rounded.

Furthermore, as a preferred embodiment, the purification device body 1 comprises: a device main housing 101, a power supply 102, a fan assembly 103, a control circuit board 104, and an air pipe 105, the fan assembly 103 is fixedly installed on one side of an interior of the device main housing 101, the control circuit board 104 is fixedly installed on the other side of the device main housing 101, the power supply 102 is fixedly installed between the fan assembly 103 and the control circuit board 104, both the power supply 102 and the fan assembly 103 are connected to the control circuit board 104, the air pipe 105 is arranged on one side other than the side on which the fan assembly 103 opposites to the power supply 102, one end of the air pipe 105 passes through the device main housing 101 and communicates with an interior of the fan assembly 103, and the other end of the air pipe 105 communicates with a breathing apparatus of a user. Furthermore, when powered by the power supply 102, the control circuit board 104 controls the fan assembly 103 to operate, so that external air sequentially passes through the filter element cover 3, the filter element 2, the fan assembly 103, and the air pipe 105, so that the filtered air is supplied to a breathing mask or other air breathing devices communicating with the air pipe 105.

Furthermore, as a preferred embodiment, the device main housing 101 comprises: an upper housing 1011 and a lower housing 1012, the upper housing 1011 is detachably installed at an upper end of the lower housing 1012, an outer edge of the upper housing 1011 matches an outer edge of the lower housing 1012, the notch 11 is formed on a side of an upper portion of the upper housing 1011, a supporting edge 1013 is provided at an inner side of the notch 11 in a horizontal direction, an outer edge of the supporting edge 1013 matches the outer edge of the filter element 2, the filter element 2 is placed on the supporting edge 1013, an upper air duct opening 1014 is formed on an inner side of the supporting edge 1013, the upper air duct opening 1014 directly faces an upper portion of the fan assembly 103, a mounting opening is disposed at one side of the lower housing 1012, and the mounting opening matches one end of the air pipe 105. Furthermore, a lower surface of the lower housing 1012 may also be provided with a clamping structure, so that users may clamp the personal air sterilization and purification device onto their pockets or bags, then it is easy for the users to carry.

Furthermore, as a preferred embodiment, the fan assembly 103 comprises: an upper sealing wall 1031, a side sealing wall 1032, and a turbo fan 1033, the turbo fan 1033 is fixedly installed on a side of an upper surface of the lower housing 1012, the side sealing wall 1032 is installed at an upper surface of the lower housing 1012 in a vertical direction, the turbo fan 1033 is enclosed in the side sealing wall 1032, the side sealing wall 1032 communicates with the mounting opening, the upper sealing wall 1031 is arranged in a horizontal direction, the upper sealing wall 1031 is fixedly installed in the side sealing wall 1032, a contour of the upper sealing wall 1031 matches a contour of the side sealing wall 1032, a lower air duct opening is formed in the upper sealing wall 1031, an inner edge of the lower air duct opening matches an outer edge of the turbo fan 1033, the lower air duct opening directly faces the upper air duct opening 1014. An air duct from the filter element 2 to the air pipe 105 is formed among the upper sealing wall 1031, the side sealing wall 1032 and the lower housing 1012.

Furthermore, as a preferred embodiment, the control circuit board 104 is further provided with a USB interface 1035, the USB interface 1035 is arranged on one side other than the side on which the control circuit board 104 opposites to the power supply, the USB interface 1035 runs through the device main housing 101, and the USB interface 1035 matches the USB connector 44. Furthermore, the power supply 102, which is provided inside the device main housing 101, powers the sterilization assembly 4.

The above descriptions are only the preferred embodiments of the invention, not thus limiting the embodiments and scope of the invention.

The invention can also be embodied in the following embodiments apart from the above-mentioned embodiments:

In a further embodiment of the present invention, the number of the sterilization units 42 is not less than six. Furthermore, the number of the sterilization units 42 is preferably six, and the sterilization units 42 are evenly arranged at an inner side of the flexible circuit board 41.

In a further embodiment of the present invention, the sterilization units 42 are ultraviolet LEDs.

In a further embodiment of the present invention, a clamping hook 45 is arranged on an outer wall of the main housing close to the USB interface 1035. One end of the USB cable 43, close to the USB connector 44, is operatively snapped onto the clamping hook 45. Furthermore, since the clamping hook 45 is made of plastics or rubbers and other materials having certain resilience or elasticity, the USB cable can be clamped inside the clamping hook 45 when the USB cable is not used to power the flexible circuit board 41, so that the USB cable can be prevented from damage due to accidental overstretching, and it is easy for the user to carry.

The above descriptions are only the preferred embodiments of the invention, not thus limiting the embodiments and scope of the invention. Those skilled in the art should be able to realize that the schemes obtained from the content of specification and drawings of the invention are within the scope of the invention.

What is claimed is:

1. A personal air sterilization and purification device, comprising: a purification device body, a filter element, a filter element cover, and a sterilization assembly, a notch being formed at an upper portion of the purification device body, a bottom of the notch being provided with a filter installation slot, a bottom of the filter installation slot communicating with an interior of the purification device body, the filter element cover being snapped onto the purification device body, the filter element cover matching the notch, a filtering space being formed between the filter element cover and the filter installation slot, the filter element being detachably installed at a bottom side of the filtering space, the filter element cover being arranged opposite to the filter element, and the sterilization assembly being fixedly installed inside the filter element cover.

2. The personal air sterilization and purification device of claim 1, wherein the filter element cover comprises: a ventilation cover and a snap-fit cover, the ventilation cover is integrated with the snap-fit cover, the ventilation cover is provided with a plurality of ventilation holes, an outer edge of the ventilation cover extends vertically and downwardly to form a first edge covering structure, the sterilization assembly is installed at an inner side of the first edge covering structure, an opening is formed at an upper portion of the snap-fit cover, the opening matches a profile of a lower end of the first edge covering structure, the first edge covering structure is disposed on the opening, an outer edge of the snap-fit cover extends vertically and downwardly to form a second edge covering structure, the second edge covering structure has an L-shaped horizontal section, an inner side of the second edge covering structure is provided with a plurality of clamping feet, and inner side of the notch is provided with a plurality of bayonets, and the filter element cover are fitted to the purification device body through the plurality of clamping feet and the plurality of bayonets.

3. The personal air sterilization and purification device of claim 2, wherein the sterilization assembly comprises: a flexible circuit board, a plurality of sterilization units, and a USB cable, the flexible circuit board is arranged along an inner edge of the first edge covering structure, the plurality of sterilization units are uniformly installed on the flexible circuit board, one end of the USB cable passes through the first edge covering structure to connect with the flexible circuit board, the other end of the USB cable is disposed at an outer side of the filter element cover, and the other end of the USB cable is provided with a USB connector.

4. The personal air sterilization and purification device of claim 3, wherein the purification device body comprises: a device main housing, a power supply, a fan assembly, a control circuit board, and an air pipe, the fan assembly is fixedly installed on one side of an interior of the device main housing, the control circuit board is fixedly installed on the other side of the device main housing, the power supply is fixedly installed between the fan assembly and the control circuit board, both the power supply and the fan assembly are connected to the control circuit board, the air pipe is arranged on one side other than the side on which the fan assembly opposites to the power supply, one end of the air pipe passes through the device main housing and communicates with an interior of the fan assembly, and the other end of the air pipe communicates with a breathing apparatus of a user.

5. The personal air sterilization and purification device of claim 4, wherein the device main housing comprises: an upper housing and a lower housing, the upper housing is detachably installed at an upper end of the lower housing, an outer edge of the upper housing matches an outer edge of the lower housing, the notch is formed on a side of an upper portion of the upper housing, a supporting edge is provided at an inner side of the notch in a horizontal direction, an outer edge of the supporting edge matches the outer edge of the filter element, the filter element is placed on the supporting edge, an upper air duct opening is formed on an inner side of the supporting edge, the upper air duct opening directly faces an upper portion of the fan assembly, a mounting opening is disposed at one side of the lower housing, and the mounting opening matches one end of the air pipe.

6. The personal air sterilization and purification device of claim 5, wherein the fan assembly comprises: an upper sealing wall, a side sealing wall, and a turbo fan, the turbo fan is fixedly installed on a side of an upper surface of the lower housing, the side sealing wall is installed at an upper surface of the lower housing in a vertical direction, the turbo fan is enclosed in the side sealing wall, the side sealing wall communicates with the mounting opening, the upper sealing wall is arranged in a horizontal direction, the upper sealing wall is fixedly installed in the side sealing wall, a contour of the upper sealing wall matches a contour of the side sealing wall, a lower air duct opening is formed in the upper sealing wall, an inner edge of the lower air duct opening matches an outer edge of the turbo fan, the lower air duct opening directly faces the upper air duct opening, and an air duct from the filter element to the air pipe is formed among the upper sealing wall, the side sealing wall and the lower housing.

7. The personal air sterilization and purification device of claim 4, wherein the control circuit board is further provided with a USB interface, the USB interface is arranged on one side other than the side on which the control circuit board opposites to the power supply, the USB interface runs through the device main housing, and the USB interface matches the USB connector.

8. The personal air sterilization and purification device of claim 3, wherein the number of the sterilization units is not less than six.

9. The personal air sterilization and purification device of claim 3, wherein the sterilization units are ultraviolet LEDs.

10. The personal air sterilization and purification device of claim 7, wherein a clamping hook is arranged on an outer wall of the main housing close to the USB interface, one end of the USB cable, close to the USB connector, is operatively snapped onto the clamping hook.
